(19) United States
(12) Patent Application Publication
(10) Pub. No.: US 2018/0112177 A1
(43) Pub. Date: Apr. 26, 2018

(54) Preparation of Microbial Cellulose

(71) Applicant: The Government of the United States of America, as represented by the Secretary of the Navy, Arlington, VA (US)

(72) Inventors: Scott Walper, Springfield, VA (US); Michael A. Daniele, Raleigh, NC (US); Jonathan D. Yuen, Washington, DC (US)

(21) Appl. No.: 15/793,175

(22) Filed: Oct. 25, 2017

Related U.S. Application Data
(60) Provisional application No. 62/413,081, filed on Oct. 26, 2016.

(51) Int. Cl. C12N 1/22; C12R 1/02; C12P 19/04; A61K 47/38

(52) U.S. Cl. CPC C12N 1/22; A61K 47/38; C12P 19/04; C12R 1/02

(57) ABSTRACT

The properties of microbial pellicles are tuned by adjusting the physical culture conditions. A culture of Gluconacetobacter xylinus can be grown in a liquid growth media having a surface exposed to air so that a basal pellicle of microbial cellulose forms on the surface. Feeding the culture by adding additional liquid growth media at the surface, thereby submerging the basal pellicle; and then allowing the culture to grow again forms a second pellicle of microbial cellulose.

### CROSS-REFERENCE TO RELATED APPLICATIONS

This application is related to commonly-owned U.S. Patent Publication 2016/0198984 and to commonly-owned U.S. Pat. No. 9,720,318.

This application claims the benefit of U.S. Provisional Application 62/413,081 filed on Oct. 26, 2016, incorporated herein by reference.

### BACKGROUND

Cellulose, a highly abundant biomass, is produced by plants and many microorganisms. Cellulose nanofibrils size, dimensions, and shape are also determined to a certain extent by the nature of the cellulose source. The degree of crystallinity of the cellulose within the organism, as well as the dimensions of the microfibrils, varies widely from species to species. Algae and tunicate cellulose microfibrils, yield nanocrystals up to several micrometers in length. In contrast, wood microfibrils, yield much shorter nanocrystals. Although chemically the same as plant cellulose, bacterial nanocellulose (BNC) is produced in fibril structure which results in unique physiochemical properties, including high porosity, tensile strength, swelling, and water vapor permeation. Accordingly, BNC has been recognized as a promising material for both biomedical and industrial barrier applications.

A prolific non-plant producer of cellulose is the gram-negative aerobe Gluconacetobacter xylinus. The cellulose produced by this bacterium is chemically identical to plant-derived cellulose but exhibits higher crystallinity, better mechanical strength, and improved purity due to the absence of hemicellulose and lignin. The cellulose pellicle of G. xylinus forms at the air/liquid interface providing an oxygen-rich and hydrated environment, while also protecting the population from UV light. The biosynthetic process involves the polymerization of glucose monomers into linear glucan chains, which upon extracellular secretion assemble into crystalline fibers. The biosynthetic process is similar or the same in various organisms, but there are some differences in the cellulose synthase complexes and export machinery that determine the size and thickness of the cellulose microfibrils, and the great interest in cellulose macromolecules is due to their crystalline orientation. The microstructures formed by the ultrafine microfibrils of bacterial cellulose have lengths varying from 1 to 10 μm and create a dense reticulated structure stabilized by various hydrogen bonds. These networks show a high index of crystallinity and a higher degree of polymerization in comparison with plant cellulose. It is of particular interest to maintain these structural characteristics to which directly contribute to the unique functional properties of the cellulose pellicles.

BNC manufacturing typically occurs in large scale plants or operations to produce BNC for food products. BNC production for food products entails the controlled growth of thick (>1 cm) pellicles. To take of advantage of the nanomaterial properties of the BNC, it must be subsequently broken down and processed. To date, many engineering applications of bacterial nanocellulose rely up the maceration of the BNC pellicle, and the subsequent incorporation of the homogenized nanofibrils into a casted film or composite. Such methods of forming thin films suffer from several shortcoming including the difficulty in adequately dispersing cellulose fibers, lack of uniformity in formed materials, and a reduction in desired physical properties (such as strength and flexibility) due to the maceration or grinding process.

A need exists for improved techniques for preparing microbial cellulose in order to enjoy the benefits of maintaining the original pellicle structure while being able to tune its properties in situ, particularly in order to prepare thin films thereof.

### BRIEF SUMMARY

In one embodiment, a method of preparing microbial cellulose includes growing a culture of Gluconacetobacter xylinus in a liquid growth media having a surface exposed to air and allowing a basal pellicle of microbial cellulose to form on the surface; then feeding the culture by adding additional liquid growth media at the surface, thereby submerging the basal pellicle; and then allowing the culture to grow, thereby forming a second pellicle of microbial cellulose, wherein the second pellicle has a thickness of about 10 μm or less as measured when the second pellicle is dried.

In a further embodiment, a microbial cellulose pellicle includes microfibrils of bacterial cellulose having lengths of from 1 μm to 10 μm, wherein the pellicle has a thickness of about 10 μm or less as measured when the pellicle is dried, and in embodiments a dried thickness of 2 μm of less.

### BRIEF DESCRIPTION OF THE DRAWINGS

FIG. 1 illustrates pellicle thickness v. growth media depth.

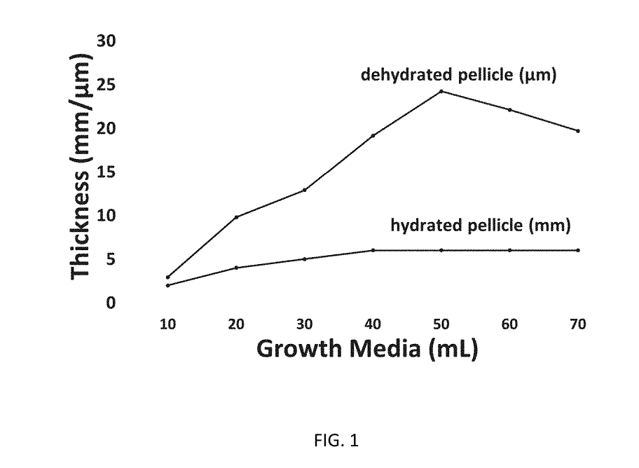

FIG. 2 illustrates glucose depletion v. growth media depth.

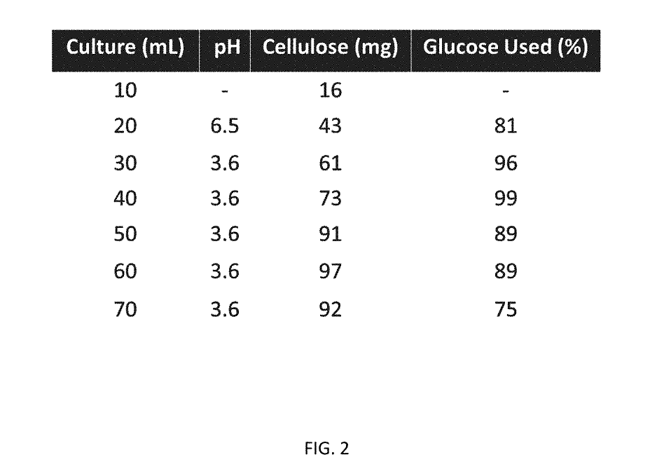

FIGS. 3A and 3B show cultures of G. xylinus. FIG. 3A shows a pellicle as a single thick layer while FIG. 3B shows stacked layers formed through iterative “feeding” cycles.

FIGS. 4A and 4B show cellulose from G. xylinus. FIG. 4A shows Hydrated pellicles harvested after 3 weeks, grown in different volumes of media.

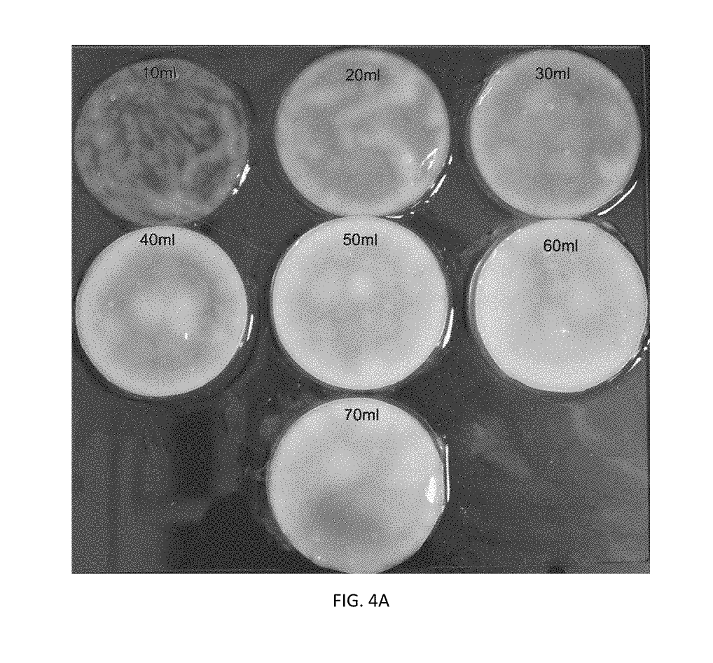

FIG. 4B shows a representative scanning electron micrograph of harvested pellicle, 30 mL growth condition. The fibril size was not altered by changes in physical growth conditions.

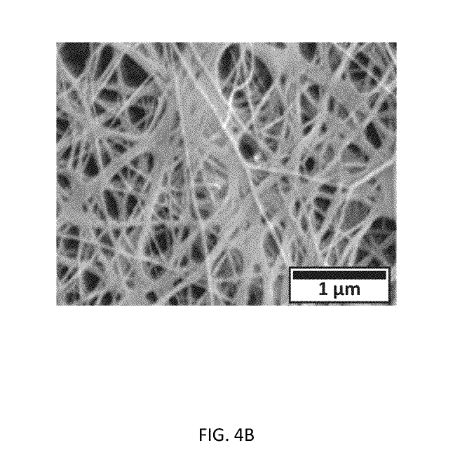

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, “nanocellulose” refers to a crystalline or semi-crystalline phase of cellulose in which one dimension, typically the diameter, is less than 100 nanometers and “microbial nanocellulose” refers to nanocellulose which is generated by the action of living bacteria.

Overview

Described herein are techniques for tuning the properties of microbial nanocellulose pellicles by adjusting the physical culture conditions. In particular, a direct method is provided to tune the thickness and morphology of microbial nanocellulose pellicles. This technology relates generally to eco-sustainable and biocompatible, fabrication of materials that can be utilized in electronics, food, and medical applications. Such results can help develop an easy method to obtain films with different nanostructures and characteristics (porosity, roughness, and crystallinity) and to develop a process in nanotechnology.

### Examples

Volume Production Method.

Gluconacetobacter xylinus can be grown in a variety of vessels as static cultures at temperatures ranging for 25-30° C., or optionally higher temperatures. “Mother cultures” are maintained in small volumes of Hestrin and Schramm medium (HS, 5-15 mL) in sterile 50 mL conical tubes. These cultures then serve as the inoculum for larger volumes/vessels. As G. xylinus does not thrive under agitation similar to other bacterial strains, methods of achieving a uniform inoculum of large-scale cultures was explored. The bacteria reside within the pellicle itself and are not easily dislodged from the pellicle. Limited success has been achieved using media collected above or below the pellicle as the inoculum. Improved efficiency was attained by vortexing the 50 mL conical tube to dislodge the pellicle from the tube side walls and free bacteria within the pellicle. Though successful, loosely associated cellulose fibrils from the basal layer of the growing pellicle were also released, complicating extraction of the bacterial suspension as this material often occluded the pipette tip and often resulted in significant variation in cell inoculum between large-scale vessels. Greater success has been achieved by vortexing the 50 mL conical tube and pellicle with 0.1 g of acid-washed 1.0 mm glass beads. To prevent cell damage, vortexing was performed for brief durations, 2-3 cycles for 5 seconds each. Using this strategy, inoculation is more consistent with all cultures forming an initial pellicle between 7-10 days. A second “feed” occurs once the initial basal pellicle has formed. Sterile HS medium is added directly to the surface of the initial pellicle. The culture is then incubated for another 7-10 days during which a second pellicle forms at the air-medium interface. It has been observed that using the two feed method forms a more consistent and uniform pellicle than other methods that have been explored to date. Once the upper pellicle has formed to the desired level, both pellicles are transferred to a new larger dish where they are rigorously washed with water to remove some of the HS medium trapped within the pellicle. The water is replaced with a 0.5M NaOH solution and the pellicles in base are transferred to a 90° C. oven for 30-60 minutes. After the base bath pellicles are again rigorously washed with water. In embodiments, the washing with water can be done before or after drying. Pellicles may discolor (yellow-orange) and discoloration can be decreased the longer the pellicles are washed in water. Typically washing is done for a minimum of 16 hours will several exchanges over that period.

Thickness Control.

Simply modifying the depth of the culture medium was effective to control thickness of the pellicle without affecting the fibril density or morphology over the majority of the pellicle. It was, however, observed that when the volume of medium was limited, bacterial growth was non-uniform as seen with the mottled pellicles (10-30 mL samples). For the 10-30 ml volumes in 100 mm culture dishes, the depth was 0.12-0.38 cm. Above the 0.4 cm depth, improved uniformity was observed if the culture was grown for extended periods of time

The transparent zones showed a reduction in fibril density but unchanged fibril morphology. Limiting the medium volume also seems to have directly affected the culture viability. The 20 mL sample did not show the characteristic drop in pH (formatting of gluconic acid) which likely means that the culture slowed or halted expansion and therefore pellicle formation. This is supported by the low percentage of glucose consumed, an indicator of culture viability. The 10 mL sample would likely have validated these observations but media was unrecoverable from the pellicle. As the volume of medium was increased the pellicles become more uniform in appearance and fibril morphology. While differences could be observed for culture volumes between 10-50 mL, pellicle morphology was unchanged with the higher volumes of medium. Pellicles harvested from the 50-70 mL volumes were of consistent density and thickness and showed similar culture viability as examined by pH measurement, glucose consumption, and total amount of cellulose produced.

Expanding upon the methods developed above, a technique was sought to produce ultra-thin (1-2 micron) layers of bacterial nanocellulose. This method relies on controlled feeding of low volumes of HS medium at defined intervals. For example, volumes of media that ranged from 6-15 ml of media added directly to the surface in a 100 mm culture dish with feeding intervals varied between feedings from 2-5 days. The optimal was found to be 8-10 ml of media at a 48 hour interval to produce uniform 1-2 micron thick pellicles.

This serial feeding process forces the cessation of cellulose production by the bacterial culture maintained in the upper most pellicle layer. Addition of fresh medium disrupts the culture/pellicle interface forcing the obligate aerobes to migrate to the new air/medium interface in order to survive. A new pellicle is then formed at this new interface. This process can be repeated indefinitely, limited only by the volume of the vessel in which the culture is maintained. Cellulose production/pellicle formation appears to halt in the submerged pellicles(s) which lends to their final uniformity.

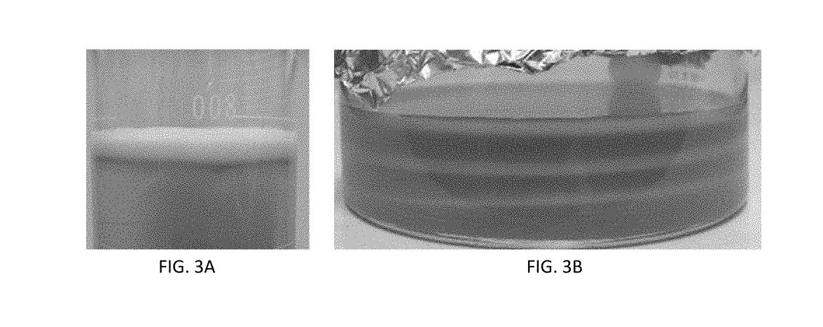

The serial feeding process can be repeated as many times as required in order to obtain a desired number of layers. For example, FIG. 3B shows a basal layer with two stacked layers above.

Pellicles are harvested with incubation in 0.5M NaOH at 90° C. for as described above. At this stage the individual pellicles layers are loosely associated with one another. The pellicles are then washed with water until an approximately neutral pH is reached. During or after water washing, the individual layers of the pellicles can be delaminated from the basal pellicle layer, manually and/or mechanically. For each feed cycle an individual pellicle will be formed and recoverable. By regulating the amount of medium added with each cycle and the time interval between the feed cycles directly correlates to the properties (thickness, fibril density, etc.) of the individual pellicles. After washing, the pellicles can be dried, typically at around 105° C.

The cellulose fibril morphology and density was characterized using atomic force microscopy (AFM). Comparison of the transparent zones to the more translucent/opaque regions of the pellicle illustrated the distribution of bacteria through culture during growth. At greater media volumes, i.e. larger media depth, the pellicles showed more consistent fibril diameter and density.

Advantages.

This technique provides the ability to tune microbial nanocellulose film thickness and nanocellulose fibril density in situ for the production of microbial nanocellulose films with thicknesses between 500 nanometers and 15 microns (as measured in a dried or dehydrated state). There is no need for substantial post-processing of raw materials to obtain desired properties. The microbial cellulose film can act as a substrate (e.g., for conformal electronics as described in U.S. Patent Publication 2016/0198984) having desirable properties such as being an oxygen barrier and/or having selective solubility. Moreover, such pellicles are more transparent than conventional films and thus suited to applications where an imperceptible is desired, such as wearable devices. Furthermore, thinner films have increased porosity which allows for more rapid wicking of materials.

### CONCLUDING REMARKS

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. A method of preparing microbial cellulose comprising:
growing a culture of Gluconacetobacter xylinus in a liquid growth media having a surface exposed to air and allowing a basal pellicle of microbial cellulose to form at the surface; then
feeding the culture by adding additional liquid growth media to the surface, thereby submerging the basal pellicle; and then
allowing the culture to grow, thereby forming a second pellicle of microbial cellulose,
wherein the second pellicle has a thickness of about 10 μm or less as measured when the second pellicle is dried.

2. The method of claim 1, further comprising an additional feeding to form a third pellicle having a thickness of about 10 μm or less as measured when the third pellicle is dried.

3. The method of claim 1, further comprising treating the second pellicle with a solution of NaOH and then washing the second pellicle with water before drying the second pellicle.

4. The method of claim 1, wherein the second pellicle comprises microfibrils of bacterial cellulose having lengths of from 1 μm to 10 μm.

5. The method of claim 1, wherein the second pellicle of microbial cellulose is composed primarily of nanocellulose.

6. A microbial cellulose pellicle comprising:
microfibrils of bacterial cellulose having lengths of from 1 μm to 10 μm, wherein the pellicle has a thickness of about 10 μm or less as measured when the pellicle is dried.

7. A microbial cellulose pellicle of claim 6, wherein the pellicle has a thickness of about 2 μm or less as measured when the pellicle is dried.

8. A microbial cellulose pellicle of claim 6, wherein the pellicle is composed primarily of nanocellulose.
